(19) United States
(12) Patent Application Publication
(10) Pub. No.: US 2007/0100335 A1
(43) Pub. Date: May 3, 2007

(54) Instrument for electrosurgical excision procedure for the uterine cervix

(75) Inventor: Nathan R. Fischer, Bloomfield, CT (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Apple Medical Corporation, Marlboro, MA (US)

(21) Appl. No.: 11/262,126

(22) Filed: Oct. 28, 2005

(51) Int. Cl. A61B 18/14

(52) U.S. Cl. 606/45

(57) ABSTRACT

A cervical biopsy instrument that includes a central shaft defining a longitudinal axis and a stop extending laterally from the shaft. The stop typically is formed in substantially the shape of a circle in a plane disposed at substantially right angles to the central axis of the shaft. The instrument also includes an electrode extending from an endocervical portion of the shaft to the stop. The stop may be formed in the shape of a complete circle, or a partial circle. Any free ends are configured to be rounded and extend inwardly toward the shaft or away from the endocervical portion to prevent the presentation of sharp edges or the like which could be caught on irregularities of the uterine cervix. The stop may be affixed to the central shaft along a tangent, or ends of the stop may be affixed to the central shaft. In another embodiment, the stop encircles or partially encircles the shaft, with the shaft extending substantially through the center of the circle. In this embodiment, either one or two electrodes may be provided. Spokes support the stop with respect to the shaft. The spokes may be formed at an acute angle with respect to the shaft to extend away from the shaft toward the endocervical end thereof. These configurations allow the instrument to be rotated in either a clockwise or a counter-clockwise direction about the central axis of the shaft without fear of catching on the uterine cervix. In another aspect of the invention, a marker is provided on the stop to indicate the location of the electrode.

### BACKGROUND OF INVENTION

1. Field of Invention This invention relates generally to an instrument for the surgical excision of tissues, more particularly to an instrument for electrosurgically excising a tissue specimen from the transformation zone of the uterine cervix.

2. Background of the Invention

Cancer of the uterine cervix, or cervical cancer, usually progresses slowly over an extended period from the first appearance of precancerous abnormalities. This gradual progression presents an opportunity for many patients to entirely avoid cervical cancer if they can benefit from preventive intervention. Even for those who do not, there is a good prognosis if the cancer is detected and treated early enough.

Before malignant cells are found, the tissues of the cervix go through changes in which abnormal cells begin to appear, initially on the epithelial tissue on the surface of the cervix. This precancerous condition is known as dysplasia or cervical intraepithelial neoplasia (CIN). CIN is a lesion of abnormal cells typically associated with the human papilloma virus (HPV). When HPV is contracted, it infects the cells of the transformation zone of the cervix where cells of the cervix actively divide and grow. As these cells grow and mature, they are pushed to the surface as new cells are produced and older, outside cells die and are shed. As the virus infects the cells and then becomes active, abnormal cells begin to be produced in the transformation zone and a lesion develops in the epithelial tissue at the surface of the cervix.

If left untreated, the cancer cells will start to grow and spread more deeply into the cervix and to surrounding areas. Approximately 30-50% of CIN conditions may progress to invasive cancer if not treated.

Removal of the lesion is an effective treatment for CIN. Although HPV remains once the CIN lesion is removed, the rate of reoccurrence is low since the removal of the transformation zone eliminates the tissue most susceptible to CIN.

Lesioned or dysplastic cells may be removed by cauterization, cryosurgery or laser surgery. A common surgical excision procedure employs loop electrosurgery, sometimes called the Loop Electrosurgical Excision Procedure or LEEP. LEEP uses a thin wire loop electrode connected to an electrosurgical generator that emits an electric current to cut away affected tissue. A low voltage and relatively high frequency electric current is emitted from the loop into the tissue cells in the immediate area of the loop wire. Consequently, the cells heat rapidly, generating steam that causes the cells to burst microscopically. Thus the tissue is divided as the wire moves through the tissue. The wire is typically swept across the projecting neck of the cervix, or ectocervix, thereby removing a thin annular slice of tissue. LEEP allows the physician to have the excised specimen analyzed by a pathology laboratory. This allows for more accurate assessment of the abnormal tissue, which will confirm either that the lesion has been completely removed with the biopsy sample, or if it has not, what further treatment may be necessary. The entire LEEP procedure usually takes less than 15 minutes and is generally very effective and well received.

One problem with LEEP is that the sample of tissue obtained may vary in amount and definition. LEEP is particularly prone to producing fragmented and burnt biopsy samples if it is necessary to make multiple passes of the loop. These problems with the samples reduce the accuracy of diagnosis. Another problem with LEEP is incomplete removal of the lesion when excising the transformation zone. This result can only worsen the patient's long-term prognosis and requires further procedures to remove the remaining abnormal cells. The converse risk is removal of too much tissue which may result in cervical stenosis or incompetent cervix and/or sterility. The primary cause of these problems is the inherent lack of stability of the loop, because it is generally on the distal end of a long handle for which there is no guiding support. This lack of stability is exacerbated because the electrode itself can move in relation to the handle under the influence of drag as the electrode passes through the cervical tissue. This compounds the difficulty of knowing exactly where the electrode is positioned at all times and adds to the difficulty of producing an accurate cut.

One solution to the problems associated with LEEP is the Fischer cone biopsy device as described in U.S. Pat. Nos. 5,554,159 and 5,403,310. The Fischer device includes an elongated insulated body member with an endocervical portion at the distal end, a contact portion at the proximal end, and a vaginal portion between the endocervical and contact portions. An insulated stop arm extends at right angles to the body member at the junction of the endocervical and vaginal portions. A wire electrode extends diagonally between the stop arm and the endocervical portion.

In use, the Fischer device is connected by means of the contact portion to an electrosurgical generator such as a blend cutting diathermy machine. The instrument is then inserted into the cervix through the vaginal canal. The vaginal canal is held open by a duckbill speculum as is well known in the art. The endocervical portion is inserted into the cervical canal to gain support for the distal end of the instrument, whereupon the wire electrode is energized through the contact portion and a conductor within the insulated body. When the energized wire contacts the cervical tissue at the ectocervix, a current passes through the wire into the patient to return through an electrode attached to a convenient part of the patient's body. This current heats the tissue cells adjacent the wire until they burst. The wire is then advanced through the cervix in a direction parallel to the longitudinal axis of the body member and the wire separates the cervical tissue. The endocervical portion extends farther into the cervical canal until the stop arm touches the ectocervix. When this position is reached, the instrument is turned one full revolution (360°) about the longitudinal axis of the body member to cut a conical tissue specimen from the transformation zone. The current is then turned off and the cut specimen is withdrawn with the instrument from the vaginal canal.

The endocervical portion extending into the cervical canal acts as a pivot about which the Fischer device is turned during the cutting operation. The pivot rotatably supports the distal end of the instrument. The arm abutting the ectocervix acts to determine the depth of cut during the cutting operation. The Fischer device has an improved accuracy over the loop electrode because the arm holds the diagonal electrode taut and straight throughout the procedure and because the electrode itself is as short as possible.

On occasion, the protruding end of the stop arm of the Fischer device, particularly a curved stop arm, has a tendency to engage or catch irregularities of the uterine cervix, thus interfering with the smooth rotation of the Fischer device about its axis. It is also desired by some physicians to be able to rotate the Fischer device about its axis in either a clockwise or counterclockwise direction without the stop arm catching on the uterine cervix.

Other related instruments are discussed in the following U.S. Pat. Nos. 6,730,085; 6,416,513; 6,309,388; 6,514,481; 6,344,026; 6,540,695; 6,659,105; 6,669,643; 6,676,658; 5,951,550; 5,676,663; and 5,616,469.

### SUMMARY OF INVENTION

This invention relates generally to endocervical excision instruments which are specifically used for the excision of a tissue specimen from the transformation zone of a uterine cervix. Those instruments comprise various new embodiments of the Fischer device described above. In particular, the various configurations of the stop on the instrument facilitate rotation of the instrument in either direction without engaging or catching irregularities of the uterine cervix which could otherwise interfere with the smooth rotation of the instrument about its axis.

In one aspect, an instrument for excision of a tissue specimen from a transformation zone of a uterine cervix is disclosed. In one embodiment of this aspect, the instrument includes an elongated shaft having an endocervical portion disposed adjacent a first end, and a contact portion disposed adjacent a second end, in which the endocervical portion is structured to be inserted into a uterine cervix. A curved stop is disposed between the endocervical portion and the contact portion of the shaft in which the stop subtends an arc of greater than 180° in a plane disposed substantially perpendicularly of the shaft. At least one wire electrode extends from the stop to the endocervical portion of the shaft. In one embodiment of this aspect, the stop subtends an arc greater than about 270°. In another embodiment, the stop subtends an arc of about 360°. In yet another embodiment, the stop is substantially circular in shape. The stop may be affixed to the shaft along a line tangent to the stop. In another embodiment, two ends of the stop may be affixed to the shaft. Alternatively, a single end of the stop may be affixed to the shaft. In yet another embodiment, the stop may include two spaced ends which are disposed at a location spaced from the shaft. In one embodiment, the two ends are rounded and extend away from the endocervical portion and toward the contact portion. In yet another embodiment in which there are two spaced ends, the ends are rounded and extend radially inwardly toward the shaft. In another embodiment in which there are two spaced ends, the stop subtends an arc of greater than 180° between the two spaced ends.

In yet another embodiment of this aspect, the shaft extends generally through a center of the stop. In this embodiment, a spoke may extend from the shaft to the stop. This embodiment may also include a second electrode extending from the endocervical portion of the shaft to the stop. In another embodiment of this aspect, a visual marker may be placed on the stop adjacent a location at which the electrode is affixed to the stop. In one variation of this embodiment, the visual marker is disposed on a side of the stop facing the contact portion.

In another aspect of this invention, an instrument for excision of a tissue specimen from a transformation zone of a uterine cervix includes an elongated shaft having an endocervical portion disposed adjacent a first end and a contact portion structured to be coupled to a source of electric current disposed at a second end, the endocervical portion being structured to be inserted into a uterine cervix. A stop may be disposed between the endocervical portion and the contact portion and is structured to abut an ectocervix. At least one wire electrode may be electrically coupled to the contact portion and extends from an attachment spot on the stop to the endocervical portion of the shaft. A visually observable marker may be disposed on the stop at the attachment spot, the marker facing the contact portion of the shaft. In one embodiment of this aspect, the marker has a color different from a color of a uterine cervix. In another embodiment, this color may be yellow.

In another aspect of this invention, an instrument for excision of a tissue specimen from a transformation zone of a uterine cervix includes an elongated shaft having an endocervical portion disposed adjacent a first end and a contact portion disposed adjacent a second end, a stop disposed between the endocervical portion and the contact portion, the stop being disposed substantially perpendicularly of the shaft, the stop being rounded and having no free ends or edges, and at least one electrode extending from the stop to the endocervical portion of the shaft. In one embodiment of this aspect, the stop has a substantially circular shape. In another embodiment of this aspect, the stop is affixed to the shaft along at least one surface of the stop. In yet another embodiment of this aspect, the stop surrounds the shaft and is coupled to the shaft by at least one spoke.

In yet another aspect of this invention, an instrument for excision of a tissue specimen from a transformation zone of a uterine cervix includes an elongated shaft having an endocervical portion disposed adjacent a first end and a contact portion disposed adjacent a second end. A stop is disposed between the endocervical portion and the contact portion, the stop being disposed substantially perpendicularly of the shaft and being rounded and subtending an arc of less than 180° centered on the shaft. This instrument also includes at least one wire electrode extending from the stop to the endocervical portion of the shaft. In one embodiment of this aspect, the instrument includes two spokes extending from the shaft to the stop. In yet another embodiment of this aspect, the stop includes two rounded ends which are curved away from the endocervical portion and toward the contact portion of the shaft. In yet another embodiment of this aspect, the stop subtends an arc of about 90°. In yet another further embodiment of this aspect, the spokes form an angle with respect to the shaft of less than 90° and extend toward the endocervical portion of the shaft.

In yet another further aspect of this invention, an instrument for excision of a tissue specimen from a transformation zone of a uterine cervix includes an elongated shaft having an endocervical portion disposed adjacent a first end and a contact portion disposed adjacent a second end in which the endocervical portion is structured to be inserted into the uterine cervix. A stop is disposed between the endocervical portion and the contact portion, and the stop includes a first end which is affixed to the shaft and a second, free end. The stop includes a laterally offset portion which is arcuate in shape and a curved portion disposed between the laterally offset portion and the second end of the stop so that the second end extends away from the endocervical portion and toward the contact portion. This embodiment further includes a wire electrode extending from the endocervical portion of the shaft to a location on the stop disposed between the laterally offset portion and the curved portion. In another embodiment of this aspect, the second end of the stop is rounded, and in yet another embodiment, the second end of the stop has an enlarged, bulbous shape.

### BRIEF DESCRIPTION OF DRAWINGS

The objects, advantages and features of this invention will be more clearly appreciated from the following detailed description, when taken in conjunction with the accompanying drawings, in which:

FIG. 1 is a perspective view of one embodiment of the present invention;

FIG. 2 is a distal end view of the embodiment of FIG. 1;

FIG. 3 is a cross-sectional side view taken along the line 3-3 of FIG. 1;

FIG. 4 is a perspective view of another embodiment of the present invention;

FIG. 5 is a perspective view of yet another embodiment of the present invention;

FIG. 6 is a is a perspective view of yet another embodiment of the present invention;

FIG. 7 is a perspective view of yet another embodiment of the present invention;

FIG. 8 is a perspective view of another embodiment of the device of FIG. 7;

FIG. 9 is a distal end view of the embodiment of FIG. 7;

FIG. 10 is a perspective view of yet another embodiment of the device of FIG. 7;

FIG. 11 is a perspective view of an alternative embodiment of the device of FIG. 10;

FIG. 12 is a perspective view of an alternative embodiment of the device of FIG. 11;

FIG. 13 is a perspective view of yet another further embodiment of the present invention;

FIG. 14 is a perspective view of an alternative embodiment of the device of FIG. 13;

FIG. 15 is a diagrammatic, perspective view of the embodiment of FIG. 1 being inserted into the vaginal canal and uterine cervix; and

FIG. 16 is a diagrammatic perspective view illustrating rotation of the embodiment of FIG. 1.

### DETAILED DESCRIPTION

The present invention relates to instruments which are modifications of the Fischer cone biopsy device, as described in U.S. Pat. Nos. 5,554,159 and 5,403,310, which are incorporated herein by reference in their entirety. In one aspect of the invention, the stop is configured to have no protruding edges or ends. The stop is configured such that the central shaft or body member may be rotated about its axis in either a clockwise or a counterclockwise direction without any protruding edges or ends on the stop that may catch on irregularities in the uterine cervix. In one embodiment of this aspect, the stop has a substantially curved shape throughout its length. Typically, in this embodiment, the stop has a substantially circular configuration. This stop may extend over a complete 360° arc, or it may extend over any arc greater than 180°. In one embodiment, a substantially circular stop is mounted to the central shaft along one edge such that the shaft forms a tangent with respect to the stop. In another embodiment, ends of the stop terminate on the shaft. In yet another embodiment, one end of the stop is affixed to the shaft, while another end of the stop is spaced from the shaft. In yet another embodiment of this aspect, the stop contains two spaced ends which are located in spaced relation with the shaft, and the arm is attached substantially along a tangent. These spaced ends typically are rounded and extend inwardly toward the shaft to avoid catching on irregularities in the uterine cervix. Each embodiment includes at least one electrode extending from the endocervical portion of the shaft to the stop. The electrode could either extend diagonally or include an angular bend between the stop and the shaft.

In another embodiment, the stop forms a complete or a partial circle and surrounds the shaft. In one embodiment of this aspect, the stop is connected to the shaft by at least one spoke. In another embodiment of this aspect, the stop is connected to the shaft by two or more spokes. The spokes may either be perpendical to the shaft or form an angle of less than 90° with respect to the shaft and extend toward the endocervical portion. A wire electrode extends between a point on the stop, and the endocervical portion of the shaft. In another embodiment, there are two wire electrodes extending between the stop and the endocervical portion. In yet another embodiment, the stop does not form a complete circle and includes two spaced, rounded ends. The two ends may extend away from the endocervical portion toward the contact portion of the shaft. The stop may subtend an arc of less than 180°, or 90° or less, centered on the shaft.

In yet another aspect of the invention, the stop may include two ends, one of which is bonded to the shaft and the other of which is rounded and extends away from the endocervical portion and toward the contact portion. In one embodiment, the stop includes an arcuate, laterally offset portion and a curved portion between the laterally offset portion and the other end. The other end may be enlarged or bulbous in another embodiment.

In another aspect of the present invention, a marker is placed on the stop at the point at which each wire electrode is connected thereto. This marker provides a visual indication to the surgeon of the location of the wire electrode.

With reference now to the drawings, and more particularly to FIG. 1 thereof, one embodiment of the instrument of this invention will now be described. Instrument 10 includes an elongated shaft 12, an electrode 14 and a stop 15. Typically, shaft 12 is elongated, is generally cylindrical in shape and has a central axis of rotation 13. Preferably, shaft 12 includes an endocervical portion 16 at one end thereof, a contact portion 18 at the end of shaft 12 opposite the endocervical portion 16 and a vaginal portion 20 between the contact portion 18 and the endocervical portion 16.

In one embodiment, stop 15 extends at a substantially right angle to axis 13 and intersects shaft 12 generally at the juncture of the endocervical portion 16 and the vaginal portion 20.

Typically, shaft 12 includes a core 22 formed of an electroconductive material, such as stainless steel. Surrounding core 22 is a coating 24 of electrically insulating material (FIG. 3). One example of such an electrically insulating material is polytetrafluoroethylene. Other examples may include any other insulating synthetic resins or rubber. Contact portion 18 remains exposed and is not coated with coating 24. Contact portion 18 typically is an extension of core 22. Typically, although not necessarily, stop 15 is formed of the same material as shaft 12, namely an inner core 21 of an electroconductive material surrounded by a coating 23 of electrically insulating material. The core 21 of stop 15 may also be stainless steel.

Electrode 14 typically is formed from a thin, electrically conductive wire which is uncoated and exposed. Electrode 14 may be formed of any electrically conductive material, such as a metal, that includes copper, steel, tungsten or the like. As shown in FIG. 1, electrode 14 may extend diagonally from the endocervical portion 16 of shaft 12 to a point on stop 15. As is shown in phantom in FIG. 3, in an alternative configuration, electrode 14A may have a squared-off configuration that includes a bend 11, intermediate stop 15 and shaft 12. Electrode 14A typically intersects shaft 12 at approximately right angles and bend 11 may form an angle somewhat greater than 90°. Bend 11 is typically permanently formed in electrode 14A. A somewhat stiffer wire, such as a tungsten wire, may be used for electrode 14A so that bend 11 retains its shape during use.

Electrodes 14 and 14A may be coupled to stop 15 at any one of several locations on stop 15. Electrode 14 may be positioned at varying angles with respect to shaft 12 as is appropriate for different clinical situations. This angle may be varied by altering the position of the location where electrode 14 is anchored to the endocervical portion of shaft 12, and/or by altering the diameter of stop 15 and/or by altering the location where electrode 14 is affixed to stop 15. Typically electrode 14 forms an acute angle greater than about 10° with respect to axis 13 of shaft 12. It also is preferred that electrode 14 not be coupled to shaft 12 adjacent ends 52 or 54. Electrode 14 is electrically coupled to core 22 of shaft 12 as well as the electrically conductive core 21 of stop 15. Electrode 14A is coupled to both core 21 and core 22 in the same manner as electrode 14. Preferably, although not necessarily, electrode 14 contacts core 22 of endocervical portion 16 at a point spaced inwardly from the free, distal end of shaft 12. In one embodiment, electrode 14 is electrically coupled to core 22 by an anchor bead 28 of electrically conductive material which also bonds electrode 14 to shaft 12. Similarly, in one embodiment, electrode 14 is coupled to core 21 of stop 15 by an anchor bead 26 of electrically conductive material which both anchors electrode 14 to stop 15 and electrically couples electrode 14 to core 21 of stop 15.

In the embodiment of FIG. 1, stop 15 has substantially the shape of a circle in a plane that typically, but not necessarily, is perpendicular to axis 13. In the embodiment of FIG. 1, stop 15 includes ends 52 and 54 which are bonded to shaft 12. Ends 52 and 54 of stop 15 may be bonded to shaft 12 such that the core 21 of stop 15 is in electrical contact with core 22 of shaft 12. Ends 52 and 54 may be affixed to shaft 12 in any conventional manner, such as by soldering, brazing, welding, gluing and the like.

Another embodiment of this aspect of the invention is illustrated in FIG. 4. Like numbers are used for like parts where appropriate. As can be seen in FIG. 4, stop 45 has substantially the shape of a circle in a plane that typically is substantially perpendicular to axis 13. In this embodiment, stop 45 is affixed to shaft 12 generally at the junction of vaginal portion 20 and endocervical portion 16 of shaft 12 at a point on the external surface of stop 45. Shaft 10 may be substantially tangent to stop 45. In this embodiment, like the embodiment of FIG. 1, the core of stop 45 is bonded to or is in electrical communication with the core of shaft 12. This embodiment may include an electrode 14 coupled to stop 45 at almost any point such that electrode 14 forms an acute angle of greater than 10° with respect to axis 13 of shaft 12. This embodiment may include a squared-off electrode like electrode 14A as shown in FIG. 3, in place of electrode 14. In all other respects, the embodiment of FIG. 4 is the same as that of FIG. 1.

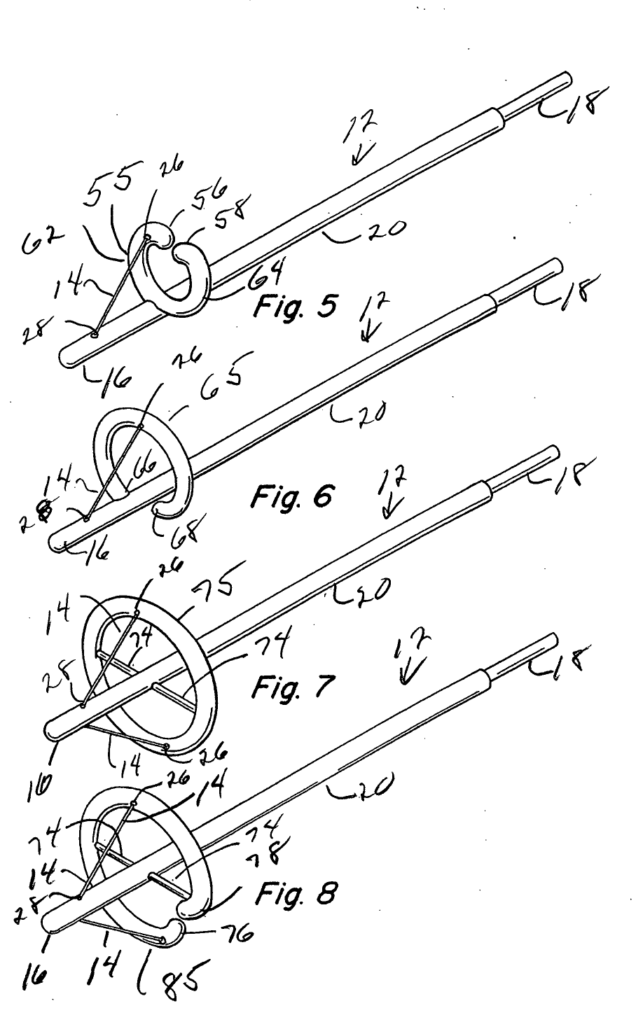

Another embodiment of this aspect is illustrated in FIG. 5. Like numbers are used for like parts where appropriate. In the embodiment of FIG. 5, stop 55 has a substantially circular shape in a plane generally perpendicular to axis 13. In this embodiment, however, stop 55 does not form an entirely closed circle. Rather, stop 55 includes spaced ends 56 and 58 which form arms 62 and 64. In this embodiment, stop 55 may be bonded to shaft 12 in the manner shown in FIG. 1 or in the manner shown in FIG. 4. The core of stop 55 is electrically coupled to the core of shaft 12. As shown in FIG. 5, typically, electrode 14 is affixed to stop 55 adjacent an end 56. However, it is to be understood that electrode 14 may be affixed to stop 55 at any other point along stop 55, so long as electrode 14 forms an angle greater than about 10° with respect to axis 13 of shaft 12. As in the embodiments of FIGS. 1 and 4, electrode 14 is bonded and electrically coupled to the core of stop 55 and to the core of shaft 12. As in the other embodiments, electrode 14 is bonded to electrocervical portion 16 at a point spaced inwardly from the distal end thereof. The embodiment of FIG. 5 may include a squared-off electrode like electrode 14A as shown in FIG. 3, in place of electrode 14.

In the embodiment of FIG. 5, typically ends 56 and 58 are spaced apart a sufficiently small distance such that when shaft 12 is rotated in either a clockwise or a counter-clockwise direction about axis 13, ends 56 and 58 do not interfere with the rotation of shaft 10 by catching irregularities of the uterine cervix. Ends 56 and 58 may be spaced apart from one another a distance such that the stop typically subtends an arc of greater than 180°, the center of the arc being axis 13. The angle subtended by the arc also may be 270° or greater. In one embodiment, ends 56 and 58 are spaced apart a distance of about 2 centimeters or less. It is to be understood, however, that this is merely one example and that other spacings are permitted so long as the stop subtends an arc of greater than 180°.

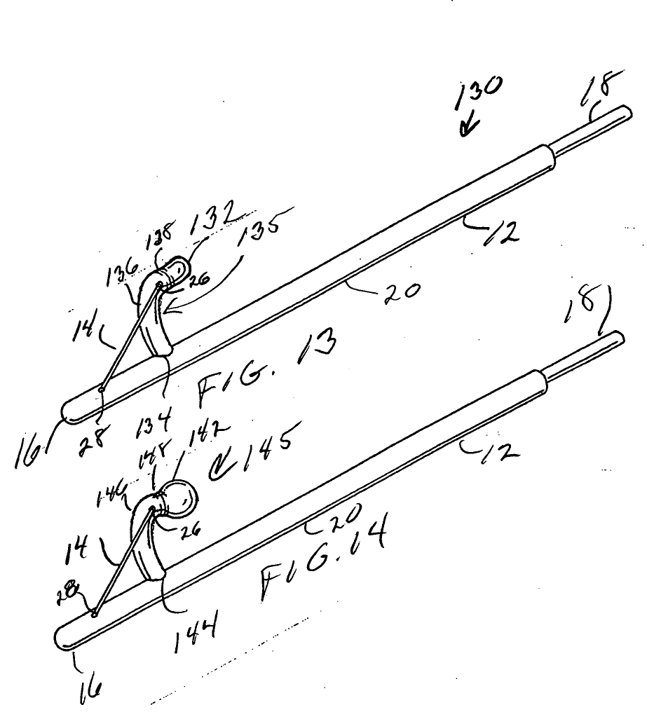

In another embodiment, ends 56 and 58 are configured to have outer surfaces which curve or slant inwardly toward shaft 12, as shown in FIG. 5. Alternatively, ends 56 and 58 may curve and extend away from endocervical portion 16 and toward contact portion 18. This configuration is shown in conjunction with the embodiments of FIGS. 11-14. Moreover, the outer surfaces of ends 56 and 58 are typically smooth or rounded without any sharp edges or ends. These features help prevent ends 56 and 58 from catching any irregularities in the uterine cervix.

Ends 56 and 58 may be positioned at almost any location on stop 55 so that resulting arms 62 and 64 of stop 55 are of almost any length. For example, as shown in FIG. 5, arms 62 and 64 may be of substantially the same length, so that the gap betweens ends 56 and 58 is substantially centered on and directly opposite the point at which stop 55 is affixed to shaft 12. However, it is to be understood that ends 56 and 58 can be positioned at a location closer to shaft 12, so that arms 62 and 64 are of differing lengths.

Another embodiment of this aspect of the invention is illustrated in FIG. 6. Like numbers are used for like parts where appropriate. In this embodiment, stop 65 includes ends 66 and 68. Stop 65 may be substantially circular in shape. End 66 is affixed to shaft 12 and its core is electrically coupled to core 22 of shaft 12. End 68, on the other hand, is spaced from shaft 12. As in the embodiment of FIG. 5, end 68 is configured so as not to catch irregularities in the uterine cervix. End 68 may accomplish this result by curving inwardly toward shaft 12, as shown in FIG. 6, or by curving away from endocervical portion 16 and toward contact portion 18 in the manner shown in FIGS. 11-14, and by being rounded and having no sharp edges or ends thereon. In this embodiment, electrode 14 typically is coupled to the core of stop 65 at a point between ends 66 and 68 such that the angle formed between electrode 14 and axis 13 of shaft 12 is an acute angle greater than about 10°. As in other embodiments, a squared-off electrode 14A shown in FIG. 3 may be used in place of electrode 14. In all other respects, the embodiment of FIG. 6 is the same as that of FIGS. 1-5.

Use of the embodiment of FIG. 1 will now be described with particular reference to FIGS. 15 and 16. Each of the devices of FIGS. 1 and 3-6 is operated in substantially the same manner, so that the following description of the use and operation of the embodiment of FIG. 1 also applies equally to the use and operation of the embodiments of FIGS. 4-6. The device of FIG. 1 is intended to be used with the uterine cervix 36, portions of which are schematically shown in FIGS. 15 and 16. Contact portion 18 is dimensioned to fit into a manipulator 30. Manipulator 30 is part of a standard electrosurgical generator (not shown). An electric current flows from manipulator 30 through uncovered contact portion 18 and through core 22 to electrode 14. A return flow of current passes through an electrode attached elsewhere to the patient's body (not shown). Shaft 12 of instrument 10 is dimensioned so that contact portion 18 is always disposed outside the vaginal canal 32 when the endocervical portion 16 is inserted into the endocervical canal 34 of the uterine cervix 36.

Endocervical portion 16 of the instrument is inserted axially through the vaginal canal 32 and into the endocervical canal 34 of the uterine cervix 36 until electrode 14 contacts an area of the ectocervix 38 which is free from all evident pathology. A current is imparted to electrode 14 through manipulator 30. Electrode 14 cuts through the tissue of the ectocervix 38 allowing the endocervical portion 16 to be advanced axially in the direction shown by the arrow in FIG. 15 into the endocervical canal 34 until stop 15 abuts the ectocervix 38, as shown in FIG. 16. In this manner, an axial cut is made in the transformation zone 40 of the uterine cervix 36 by electrode 14. Because stop 15 is seated stably on ectocervix 38 and the electrode 14 is taut, the physician has good control of the cutting action and the extent of penetration is limited.

At this point, current to electrode 14 may be discontinued to permit preparation of the next step. Once preparation is completed, current may again be imparted to electrode 14. The instrument is rotated through one full 360° revolution in one direction about its axis 13 as shown by the arrows in FIG. 16. Because there are no edges that would catch irregular portions of the uterine cervix, the instrument may be rotated in either a clockwise or a counter-clockwise direction. Because rotation takes place with the endocervical portion 16 in the endocervical canal 34 and with the stop 15 abutting the ectocervix 38, the instrument is stabilized, allowing for a tissue specimen 42 to be excised from the transformation zone 40 of the uterine cervix 36.

Once the excision is complete, current is discontinued to electrode 14 and the instrument is withdrawn from the endocervical canal 34 and the vaginal canal 32, simultaneously withdrawing the tissue specimen 42 along with it. The tissue specimen 42 typically will be conical in shape if an electrode 14 is employed. If a squared-off electrode 14A is employed, tissue specimen 42 will have a configuration approximating that of a truncated cone. In either case, a defined, controlled amount of cervical tissue is provided to make pathological interpretation easier and more reliable.

It should be understood that if only a wedge-shaped specimen is desired, the specimen may be cut by limiting the extent of rotation of the shaft 12 to an angle of less than 360°. Thereafter, the instrument is slowly axially withdrawn from the endocervical canal 34 while power is still applied to electrode 42 to cut the other end of the wedge-shaped section.

The current employed for the foregoing excision process is one appropriate for cutting and coagulation. Typically, an output power in the range of 50-70 watts is suitable.

In the embodiments of FIGS. 1 and 3-6, shaft 12 is typically about 120-140 millimeters in length. One preferred length is about 130 millimeters. Endocervical portion 16 may be about 18-22 millimeters in length, with about 20 millimeters being a suitable length. Vaginal portion 20 typically is in the range of about 80-100 millimeters in length with a suitable length being about 90 millimeters. Contact portion 18 typically is about 8-12 millimeters in length although it may be as long as about 20 millimeters. Stops 15, 45, 55 and 65 typically have a diameter of about 15-20 millimeters. Electrodes 14 and 14A typically are attached to shaft 12 at a location spaced inwardly from the distal end thereof a distance of about 2 to about 5 millimeters.

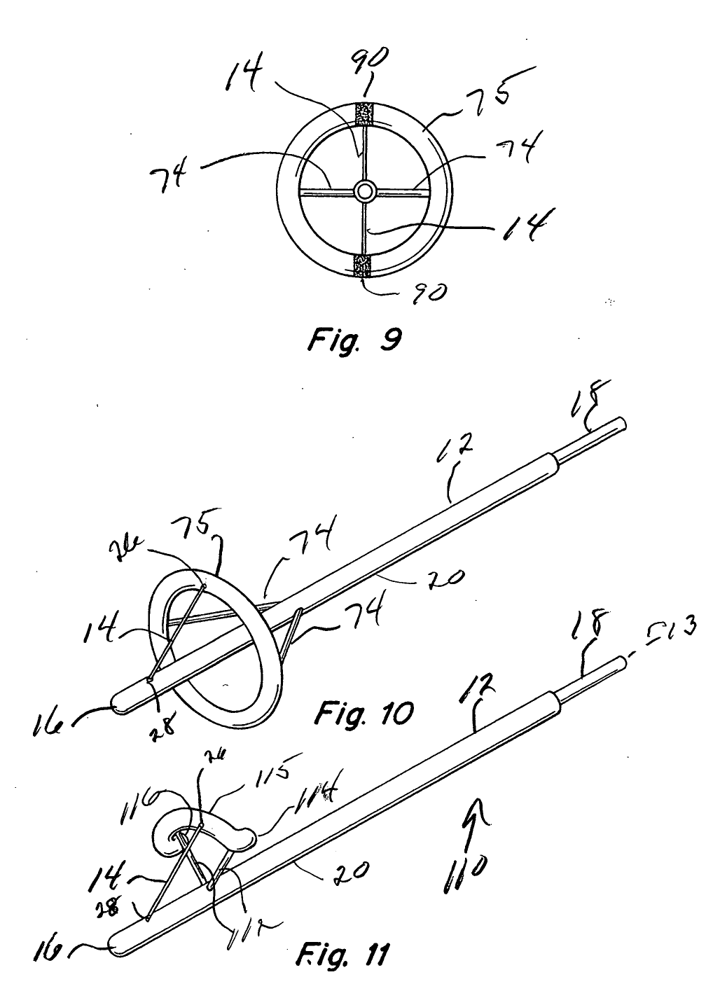

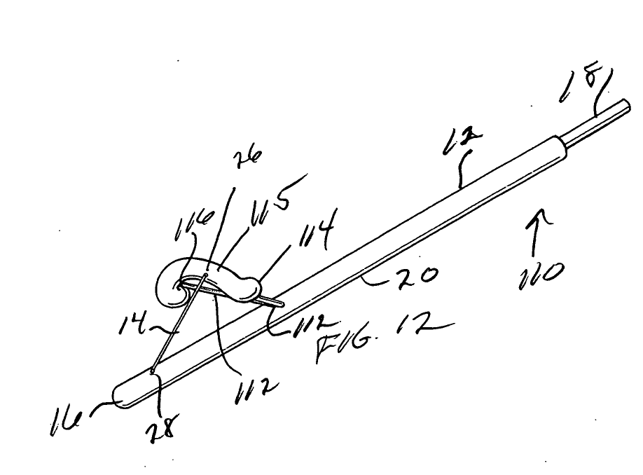

Another aspect of this invention will now be described with respect to FIGS. 7, 8, 10, 11 and 12. Like numbers will be used for like parts where appropriate. Instrument 70 of FIG. 7 includes a shaft 12 which has an endocervical portion 16 at one end, contact portion 18 at the other end, and a vaginal portion 20 therebetween. A stop 75 is disposed approximately at the juncture of the endocervical portion 16 and the vaginal portion 20. Stop 75 is curved, and in one embodiment, may be formed as a circle in a plane generally perpendicular to axis 13 of shaft 12. In the embodiment of FIGS. 7 and 10, stop 75 may form a complete circle which surrounds shaft 12. Typically, although not necessarily, shaft 12 is disposed at the center of the circle formed by stop 75. Stop 75 is supported with respect to shaft 12 by one or more spokes 74 which extend from shaft 12 to stop 75. Two spokes 74 are illustrated in FIGS. 7, 8 and 10. Spokes 74 may extend at a substantially perpendicular angle with respect to shaft 12, as shown in FIG. 7, or spokes 74 may form an acute angle with respect to shaft 12, pointing toward endocervical portion 16 and away from contact portion 18, as shown in FIG. 10. Spokes 74 typically are formed of a material which is sufficiently rigid to maintain the spacing between stop 75 and shaft 12 and to prevent rotation of stop 75 about shaft 12. A typical example of a suitable material is stainless steel. Spokes 74 provide an electrical connection between stop 75 and manipulator 30. Stop 75, as with stop 15, is typically formed of a conductive core material surrounded by a non-conductive layer. This embodiment may include at least one electrode 14, as illustrated in FIG. 10, or as illustrated in FIG. 7, two electrodes 14. Each electrode 14 extends from a point spaced inwardly from the distal end of the endocervical portion 16 of shaft 12 to a point on stop 75. If two electrodes are used, typically they are diametrically opposed about shaft 12 or are separated by an arc of 180° centered on axis 13. A suitable bead 28 may be utilized to bond each electrode 14 to shaft 12 and to electrically couple electrode 14 to the core of shaft 12. Similarly, an anchor bead 26 of electrically conductive material which is in electrical contact with the core of stop 75 may also be used to bond each electrode 14 to stop 75. It will be appreciated that, as with the other embodiments, squared-off electrodes like electrode 14A shown in FIG. 3 may be used in place of diagonally extending electrodes 14.

Preferably, spokes 74 are positioned so that they are offset radially on shaft 12 with respect to electrodes 14. As shown in the example of FIG. 7, spokes 74 form an angle of about 90° with respect to electrodes 14. In this way, spokes 74 do not obscure the surgeon's view of electrodes 14 through stop 75.

Since stop 75 forms a complete circle, there are no sharp edges or ends or other portions thereof which would inadvertently catch the irregular portions of the uterine cervix. Therefore, complete and smooth rotation of shaft 12 about axis 13 is permitted.

In the embodiment illustrated in FIG. 8, stop 85 may not be formed as a complete circle. Stop 85 of FIG. 8 has a gap between ends 76 and 78 disposed somewhere along the circumference of stop 85. Preferably, ends 76 and 78 are configured like ends 56 and 58 of FIG. 5, i.e., they are rounded and curve inwardly toward shaft 12, or curve and extend toward contact portion 18 and away from endocervical portion 16. Ends 76 and 78 preferably have no sharp edges or ends so that they do not catch irregular portions of the uterine cervix. Stop 85 typically subtends an arc greater than 180°, and typically greater than 270°, the arc being centered on axis 13. In all other respects, the embodiment of FIG. 8 is like that of FIG. 7.

In the embodiment of FIG. 10, there is one electrode 14 and two spokes 74. In this embodiment, stop 75 forms a complete circle. Spokes 74 form an acute angle with respect to shaft 12 and typically point or extend away from shaft 12 toward endocervical portion 16 and away from contact portion 18. Typically, the angle formed between spokes 74 and shaft 12 may range from about 10° to about 80°. One preferred angle is about 45°. This configuration minimizes contact between spokes 74 and cervix 36, to minimize the likelihood that spokes 74 would catch on the cervix and interfere with the smooth rotation of shaft 12 during surgery. In other respects, the embodiment of FIG. 10 is like that of FIG. 7.

Another embodiment of this aspect of the invention is illustrated in FIG. 11. Like numbers are used for like parts where appropriate. Instrument 110 includes a shaft 12 which has an endocervical portion 16 at one end, contact portion 18 at the other end, and a vaginal portion 20 therebetween. A stop 115 is disposed approximately at the juncture of the endocervical portion 16 and the vaginal portion 20. Stop 115 is curved, and in one embodiment, may be formed as a partial circle in a plane generally perpendicular to axis 13 of shaft 12. However, stop 115 need not be in a plane perpendicular to axis 13. Preferably, stop 115 subtends an arc which is centered along axis 13. Stop 115 typically subtends an arc less than 270°, and preferably less than 180°. A typical arc subtended by stop 115 is approximately 90°. Stop 115 is supported with respect to shaft 12 by one or more spokes 112. Spokes 112 extend from shaft 12 at substantially an angle of 90° with respect to shaft 12. In the particular embodiment of FIG. 11, two spokes 112 are illustrated. Spokes 112 are formed of the same material as spokes 74. Similarly, stop 115 is formed of the same material as stop 15. Moreover, shaft 12 is substantially identical to shaft 12 of FIG. 1. The embodiment of FIG. 11 also includes one electrode 14 which extends from a point spaced inwardly from a distal end of the endocervical portion 16 of shaft 12 to a point on stop 115. Preferably, electrode 14 is offset from spokes 112 to allow the surgeon to view electrode 14. In the embodiment shown in FIG. 11, electrode 14 is substantially centered between the points at which spokes 112 are attached to stop 115. A suitable anchor bead 28 may be utilized to bond electrode 14 to shaft 12 and to electrically couple electrode 14 to the core thereof. Similarly, an anchor bead 26 of electrically conductive material which is in electrical contact with the core of stop 115 may also be used to bond electrode 14 to stop 115. It will be appreciated that, as with the other embodiments, a squared-off electrode, like electrode 14A shown in FIG. 3, may be used in place of a diagonally-extending electrode 14 as shown in FIG. 11.

Stop 115 includes ends 116 and 114. Ends 116 and 114 may be rounded and extend inwardly like ends 56 and 58 of the embodiment of FIG. 5. In an alternative embodiment, as illustrated in FIG. 11, ends 114 and 116 are again rounded, but curve and extend generally axially away from endocervical portion 16 and toward contact portion 18. Since ends 114 and 116 extend away from endocervical portion 16, in use, these ends 114 and 116 also extend away from the uterine cervix 36. In this way, ends 114 and 116 are less likely to engage a portion of the uterine cervix 36 which could interfere with the free rotation of shaft 12 about its axis 13.

FIG. 12 illustrates an alternative embodiment of FIG. 11 and like numbers are used for like parts where appropriate. FIG. 12 is substantially identical to that of FIG. 11 in all respects except that spokes 112 form an acute angle with respect to shaft 12. Spokes 112 typically form an angle of between 10° and 80° with respect to shaft 12. One typical angle is 45°. Spokes 112 point or extend away from shaft 12 and away from contact portion 18 and toward endocervical portion 16. This angular configuration minimizes contact between uterine cervix 36 and spokes 112 to minimize the likelihood that spokes 112 could catch on the uterine cervix and interfere with the free rotation of shaft 12.

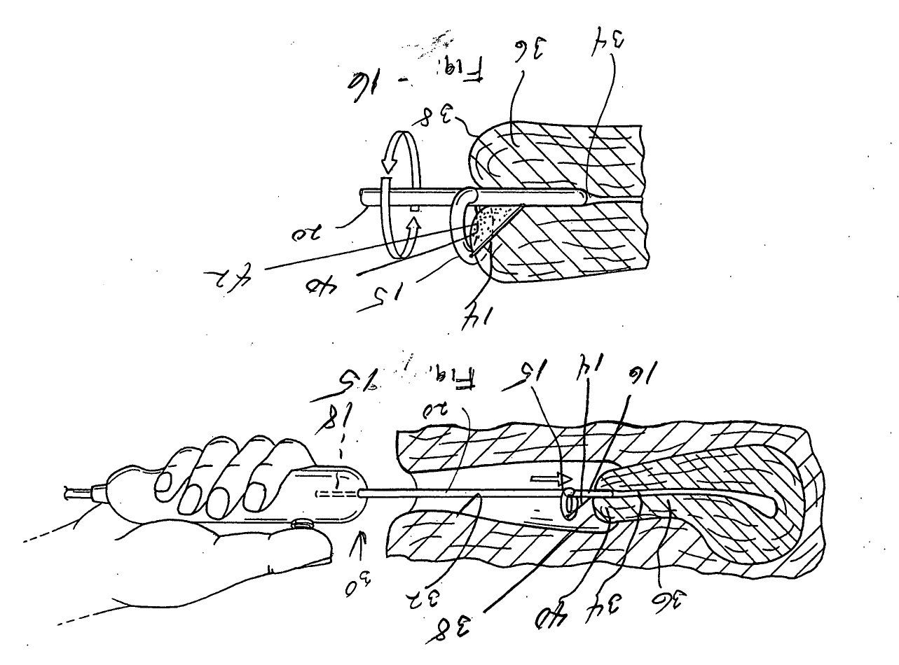

Yet another aspect of this invention will now be described with respect to FIGS. 13 and 14. Like numbers will be used for like parts where appropriate. Instrument 130 of FIG. 13 includes an electrode 14 and a shaft 12 which has an endocervical portion 16 at one end, a contact portion 18 at the other end, and a vaginal portion 20 therebetween. A stop 135 is disposed approximately at the juncture of the endocervical portion 16 and the vaginal portion 20. Stop 135 is bonded at end 134 directly to shaft 12 so that the core of stop 135 is affixed to and is in electrical contact with the core of shaft 12. Portion 136 of stop 135 between ends 132 and 134 typically is laterally offset from the longitudinal plane extending through electrode 14 and shaft 12. As a result, the physician can see most of the cervical area which is being cut by electrode 14 and which would otherwise be obscured by stop 135. Portion 136 may be arcuate in shape and have either a small or a large radius of curvature. Electrode 14 extends from a location on stop 135 between portion 136 and curve 138 to endocervical portion 16 of shaft 12 at a point spaced from the distal end thereof. It should be understood that while electrode 14 is shown as being linear and extending diagonally from stop 135 to endocervical portion 16, electrode 14 may be similar to electrode 14A of FIG. 3 and have a squared-off configuration. One end of electrode 14 may be bonded by an anchor bead 26 of electrically conductive material to the core of shaft 12, and the other end of electrode 14 may be bonded by a bead 28 of an electrically conductive material to stop 135 and is in electrical contact with the core of stop 135.

Stop 135, in this embodiment, typically includes curve 138 just beyond the point at which electrode 14 is attached to stop 135, but before end 132. Curve 138 causes end 132 to extend away from endocervical portion 16 and toward contact portion 18. Curve 138 should be smooth and rounded so that no sharp ends or edges are provided. In this way, end 132 extends away from uterine cervix 36 so as not to be inadvertently caught on uterine cervix 36 during use. End 132 is also smoothly rounded to avoid any sharp ends or edges.

An alternative embodiment of this aspect is shown in FIG. 14. Like numbers are used for like parts where appropriate. FIG. 14 has a stop 145 which includes ends 142 and 144. End 144 is bonded to shaft 12 and is in electrical communication with the core of shaft 12. Stop 145, like stop 135, has a portion 146 which is laterally offset from the longitudinal plane extending through electrode 14 and shaft 12, to allow the surgeon to see the cervical area during surgery. Stop 145 includes curve 148 beyond the point where electrode 14 is bonded to the core of stop 145 by bead 26. End 142 also extends away from endocervical portion 16 and toward contact portion 18. The primary difference between the embodiment of FIG. 14 and that of FIG. 13 is that the embodiment of FIG. 14 includes an end 142 which is slightly enlarged or is bulbous. The substantially spherical shape of end 142 assures that there are no sharp edges or ends which could catch on the uterine cervix. In all other respects, the embodiment of FIG. 14 is substantially identical to that of FIG. 13.

Use and operation of the embodiments of FIGS. 13 and 14 is substantially identical to that as described with respect to the embodiments of FIGS. 1 and 3-6, and need not be described further herein.

Operation of the embodiments of FIGS. 7, 8, 10, 11, 12, 13 and 14 is substantially identical to the operation of the embodiments of FIGS. 1 and 3-6 as illustrated with respect to FIGS. 15 and 16. The only difference of significance in the operation of the embodiments of FIGS. 7 and 8 is that because two electrodes 14 are provided, the physician only needs to rotate shaft 12 through 180°. Shaft 12 may be rotated in either a clockwise or a counterclockwise direction. It is to be understood that in FIGS. 10, 11, 12, 13 and 14, since only one electrode 14 is provided, shaft 12 may be rotated a full 360° in either a clockwise or a counterclockwise direction. Because rotation takes place with the endocervical portion 16 in the endocervical canal 34 and with stop 75, 85, 115, 135 or 145 abutting the ectocervix 38, the instrument is stabilized, allowing for a suitably shaped tissue specimen to be excised from the transformation zone 40 of the uterine cervix 36. The tissue specimen will be conically shaped if diagonal electrodes, as shown in FIGS. 7, 8, 10, 11, 12, 13 and 14 are used. If a squared-off electrode, like that shown in FIG. 3 is used, a truncated cone specimen will be obtained.

As previously discussed, if only a wedge shaped specimen is desired, shaft 12 may be rotated through an arc less than 180° for two electrodes 14, as shown in FIGS. 7 and 8, or an arc less than 360° for one electrode 14 as shown in FIG. 10. The instrument is then slowly withdrawn in an axial direction to cut the end of the section.

In a typical embodiment of FIGS. 7, 8, 10, 11, 12, 13 and 14, shaft 12, endocervical portion 16, vaginal portion 20, and contact portion 18 all have about the same dimensions as those set forth above for the embodiments of FIGS. 1 and 3-6. Typically, stops 75, 85, 115, 135 and 145 have a diameter of between about 20 and about 40 millimeters, with about 30 millimeters being a suitable diameter. Electrodes 14 are positioned on shaft 12 in substantially the same locations at substantially the same angles as the electrodes 14 in the embodiments of FIGS. 1 and 3-6.

In each of the embodiments of FIGS. 1, 3-8, 10, 11, 12, 13 and 14, the surgical procedure may be viewed through the space between respective stops 15, 45, 55, 65, 75, 85, 115, 135 and 145 and shaft 12. As a result, the surgeon can see most of the cervical area which is being cut by the electrode 14 or 14A. Nonetheless, once electrode 14 or 14A is embedded in the cervical tissue which is being cut, electrode 14 or 14A is no longer visible to the surgeon. Therefore, the exact location of the electrode is not always easily determined by the surgeon once the cutting operation begins.

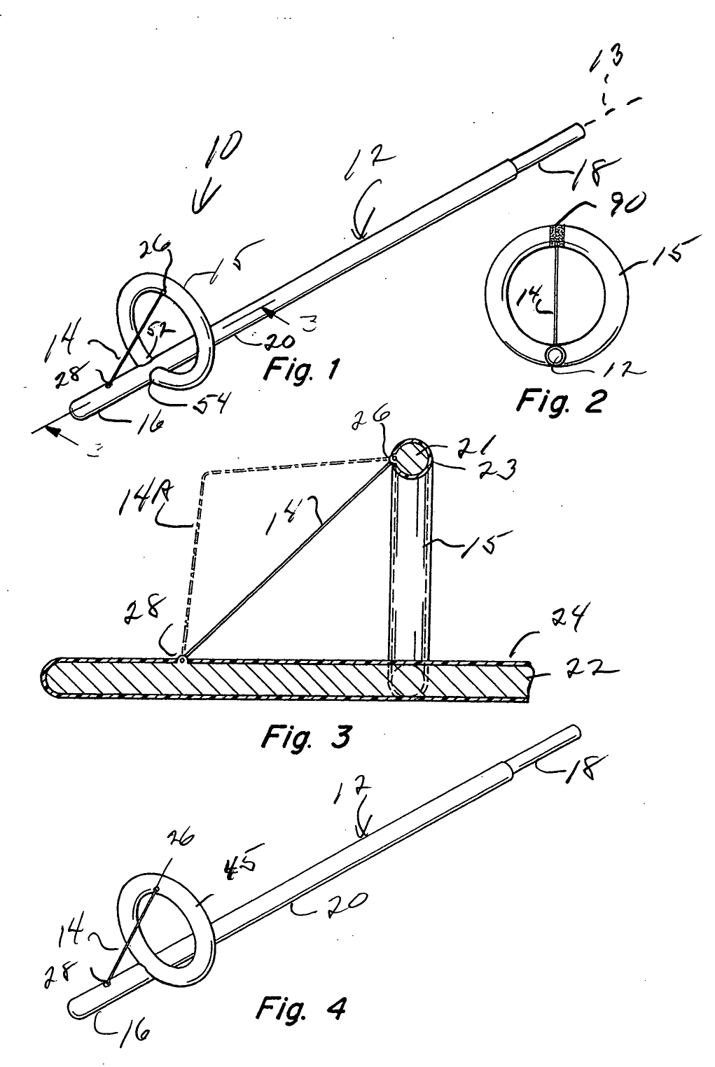

In another aspect of the invention, a marker may be provided on the stop to identify the location of the electrode(s) 14 or 14A. One embodiment of this aspect is illustrated in FIG. 2 with respect to the invention of FIG. 1. Visual marker 90 is positioned at least on the side of stop 15 facing contact portion 18 and facing away from the endocervical portion 16. Marker 90 is located directly opposite the point on stop 15 at which electrode 14 or 14A is attached to stop 15. Since the surgeon knows that the other end of electrode 14 or 14A is affixed to shaft 12, using marker 90 the surgeon can readily locate the entire length of electrode 14 or 14A, even if electrode 14 or 14A is buried in tissue. Marker 90 may be anything that provides to the surgeon a visual indication of the end of electrode 14 or 14A. In one example, marker 90 is a stripe of paint in a readily visually recognized color that contrasts with the colors present in the uterine cervix 36. One example is yellow. Other examples include white or gold. In another example, marker 90 may be a visually observable bump formed on stop 15. In yet another example, marker 90 may be a reflective or sparkling coating which allows a limited amount of light to be reflected back to the surgeon. Another example includes a wrapping of wire about stop 15.

Another embodiment of this aspect is illustrated in FIG. 9. FIG. 9 shows similar markers 90 for the embodiment of FIG. 7. Since there are two electrodes 14 or 14A in FIG. 7, there typically would be two markers 90 as shown in FIG. 9, providing an indication of the end of each electrode 14 or 14A. Markers 90 in FIG. 9 may be identical to markers 90 of FIG. 2. Since the surgeon knows that the other end of each electrode 14 or 14A is affixed to endocervical portion 16 of shaft 12, these markers 90 permit the surgeon to visually locate both electrodes 14 or 14A.

It will be appreciated that because of the substantially circular configuration of stops 15, 45, 55, 65, 75, 85, 115, 135 and 145 and the rounded ends of stops 55, 65, 85, 115, 135 and 145, there are no sharp edges or ends that could be caught on any irregularities of the uterine cervix. As a result, the shapes of these stops allow them to pass smoothly over any irregularities permitting a more precise and error-free surgical excision. Moreover, in the embodiment of FIG. 7, when two electrodes 14 are employed, the instrument need only be rotated through 180°, allowing an easier and more precise surgical incision without concern that movement of the surgeon's fingers, which may be required to rotate the instrument through an entire 360°, would cause any problems with the surgical procedure. Furthermore, the provision of an open circular shape for the stop allows a surgeon to clearly view the procedure. Finally, the provision of markers on the side of the stop facing the surgeon permits the surgeon to easily locate the electrode(s) at all times during the surgical procedure.

Modifications and improvements will occur within the scope of this invention to those skilled in the art. The above description is intended to be exemplary only, the scope of the invention being defined by the following claims and their equivalents.

What is claimed is:

1. An instrument for excision of a tissue specimen from a transformation zone of a uterine cervix comprising:
an elongated shaft having an endocervical portion disposed adjacent a first end and a contact portion disposed adjacent a second end, said endocervical portion being structured to be inserted into a uterine cervix;
a curved stop disposed between said endocervical portion and said contact portion of said shaft, said stop subtending an arc of greater than 180° in a plane disposed substantially perpendicularly of said shaft; and
at least one wire electrode extending from said stop to said endocervical portion of said shaft.

2. The instrument as recited in claim 1 wherein said stop subtends an arc greater than about 270°.

3. The instrument as recited in claim 2 wherein the stop subtends an arc of 360°.

4. The instrument as recited in claim 1 wherein said stop is substantially circular in shape.

5. The instrument as recited in claim 1 wherein the stop is affixed to the shaft along a line tangent to the stop.

6. The instrument as recited in claim 1 wherein said stop has two spaced ends.

7. The instrument as recited in claim 6 wherein both of said ends are affixed to said shaft.

8. The instrument as recited in claim 6 wherein only one of said ends is affixed to said shaft.

9. The instrument as recited in claim 6 wherein said two ends are disposed at a location spaced from said shaft.

10. The instrument as recited in claim 9 wherein said stop is affixed to said shaft along a tangent to said stop.

11. The instrument as recited in claim 9 wherein said two ends are rounded and extend away from said endocervical portion and towards said contact portion.

12. The instrument as recited in claim 9 wherein said two ends are rounded and extend radially inwardly toward said shaft.

13. The instrument as recited in claim 5 wherein said stop subtends an arc of greater than 180° between said two spaced ends.

14. The instrument as recited in claim 1 wherein said shaft extends generally through a center of said stop.

15. The instrument as recited in claim 14 further comprising at least one spoke extending from said shaft to said stop.

16. The instrument as recited in claim 15 wherein said spoke forms an acute angle with respect to said shaft and extends toward said endocervical portions.

17. The instrument as recited in claim 14 further comprising a second electrode extending from said endocervical portion of said shaft to said stop.

18. The instrument as recited in claim 1 further comprising a visual marker disposed on said stop adjacent a location at which said electrode is affixed to said stop.

19. The instrument as recited in claim 18 wherein said visual marker is at least disposed on a side of said stop facing said contact portion.

20. An instrument for excision of a tissue specimen from a transformation zone of a uterine cervix comprising:
an elongated shaft having an endocervical portion disposed adjacent a first end and a contact portion structured to be coupled to a source of electric current disposed at a second end, said endocervical portion being structured to be inserted into a uterine cervix;
a stop disposed between said endocervical portion and said contact portion and being structured to abut an ectocervix;
at least one wire electrode electrically coupled to said contact portion and extending from an attachment spot on said stop to said endocervical portion of said shaft; and
a visually observable marker disposed on said stop at said attachment spot, said marker facing said contact portion of shaft.

21. The instrument as recited in claim 20 wherein said marker has a color different from a color of a uterine cervix.

22. The instrument as recited in claim 21 wherein said marker includes a yellow color.

23. An instrument for excision of a tissue specimen from a transformation zone of a uterine cervix comprising:
an elongated shaft having an endocervical portion disposed adjacent a first end and a contact portion disposed adjacent a second end of said shaft;
a stop disposed between said endocervical portion and said contact portion, said stop being disposed substantially perpendicularly of said shaft, said stop being rounded and having no free ends; and
at least one wire electrode extending from said stop to said endocervical portion of said shaft.

24. The instrument as recited in claim 23 wherein said stop has substantially the shape of a circle.

25. The instrument as recited in claim 24 wherein said stop is affixed to said shaft along at least one surface of said stop.

26. The instrument as recited in claim 24 wherein said stop at least partially surrounds said shaft and is coupled to said shaft by at least one spoke.

27. An instrument for excision of a tissue specimen from a transformation zone of a uterine cervix comprising:
an elongated shaft having an endocervical portion disposed adjacent a first end and a contact portion disposed adjacent a second end of said shaft;
a stop disposed between said endocervical portion and said contact portion, said stop being rounded and subtending an arc centered along said shaft so that said arc at least partially surrounds said shaft; and
at least one wire electrode extending from said stop to said endocervical portion of said shaft.

28. The instrument as recited in claim 27 further comprising at least one spoke connecting said stop to said shaft.

29. The instrument as recited in claim 27 wherein said stop subtends an arc of less than 180°.

30. The instrument as recited in claim 28 wherein said stop subtends an arc of about 90°.

31. The instrument as recited in claim 27 wherein said stop includes two ends, said two ends being curved and extending away from said endocervical portion and toward said contact portion.

32. The instrument as recited in claim 29 wherein said spoke forms an acute angle with respect to said shaft and extends toward said endocervical portion.

33. The instrument as recited in claim 27 wherein said stop subtends an arc of about 360°.

34. An instrument for excision of a tissue specimen from a transformation zone of a uterine cervix comprising:
an elongated shaft having an endocervical portion disposed adjacent a first end and a contact portion disposed adjacent a second end, said endocervical portion being structured to be inserted into the uterine cervix;
a stop disposed between said endocervical portion and said contact portion of said shaft, said stop having a first end affixed to said shaft in electrical contact therewith and a second, free end, said stop having an arcuate, laterally offset portion disposed between said first end and said second end, said stop having a curved portion disposed between said laterally offset portion and said second end, said second end extending away from said endocervical portion and toward said contact portion; and
at least one wire electrode extending from a location on said stop between said laterally offset portion and said curved portion to said endocervical portion of said shaft.

35. The instrument as recited in claim 34 wherein said second end of said stop is rounded.

36. The instrument as recited in claim 35 wherein said second end has an enlarged, bulbous shape.
